(19) United States
(12) Patent Application Publication
(10) Pub. No.: US 2018/0125460 A1
(43) Pub. Date: May 10, 2018

(54) METHODS AND SYSTEMS FOR MEDICAL IMAGING SYSTEMS

(71) Applicant: General Electric Company, Schenectady, NY (US)

(72) Inventors: Christian Fritz Perrey, Zipf (AT); Walter Duda, JR., Zipf (AT)

(21) Appl. No.: 15/343,404

(22) Filed: Nov. 4, 2016

(51) Int. Cl. A61B 8/00; A61B 8/08; A61B 8/13

(52) U.S. Cl. CPC A61B 8/54; A61B 8/4494; A61B 8/13; A61B 8/5207; A61B 8/461

(57) ABSTRACT

Methods and systems are provided for selecting a two dimensional (2D) scan plane. The methods and systems acquire ultrasound data along first and second 2D planes from a matrix array probe. The second 2D plane includes an anatomical structure. The first 2D plane extending along the azimuth direction and the second 2D plane extending along the elevation direction. The systems and methods further identify when the anatomical structure is symmetric along the second 2D plane with respect to a characteristic of interest, and select select ultrasound data along the first 2D plane when the anatomical structure is symmetric.

### FIELD

Embodiments described herein generally relate to methods and systems for medical imaging systems, such as for selecting a two dimensional (2D) scan plane.

### BACKGROUND OF THE INVENTION

Diagnostic medical imaging systems typically include a scan portion and a control portion having a display. For example, ultrasound imaging systems usually include ultrasound scanning devices, such as ultrasound probes having transducers that are connected to an ultrasound system to control the acquisition of ultrasound data by performing various ultrasound scans (e.g., imaging a volume or body). The ultrasound systems are controllable to operate in different modes of operation to perform the different scans. The signals received at the probe are then communicated and processed at a back end.

Selecting two dimensional (2D) scan planes is challenging for users of conventional ultrasound imaging systems. The 2D scan planes, such as representing a mid-sagittal plane of a patient, are utilized for developmental ultrasound scans, for example for fetal biometry measurements. Conventional ultrasound imaging systems identify the mid-sagittal plane by identifying symmetry of anatomical structures within the ultrasound image, for example, utilizing machine learning algorithms. However, any tilts and/or shifts (e.g., along the elevation plane) of the ultrasound probe during the scan shifts the 2D scan plane away from the mid-sagittal plane. Additionally, tilting and/or shifts of the ultrasound probe during the scan shifts the symmetry of anatomical structures along the 2D scan plane thereby resulting in inaccurate results from the machine learning algorithms.

### BRIEF DESCRIPTION OF THE INVENTION

In an embodiment a system (e.g., an ultrasound imaging system) is provided. The system includes a matrix array probe including a plurality of transducer elements arranged in an array with an elevation direction and an azimuth direction. The system further includes a controller circuit. The controller circuit is configured to control the matrix array probe to acquire ultrasound data along first and second two dimensional (2D) planes. The second 2D plane including an anatomical structure. The first 2D plane extends along the azimuth direction and the second 2D plane extends along the elevation direction. The controller circuit is further configured to identify when the anatomical structure is symmetric along the second 2D plane with respect to a characteristic of interest and select ultrasound data along the first 2D plane when the anatomical structure is symmetric.

In an embodiment a method (e.g., a method for selecting a two dimensional (2D) scan plane) is provided. The method includes acquiring ultrasound data along first and second 2D planes from a matrix array probe. The second 2D plane includes an anatomical structure. The first 2D plane extending along the azimuth direction and the second 2D plane extending along the elevation direction. The method further includes identifying when the anatomical structure is symmetric along the second 2D plane with respect to a characteristic of interest. The method further includes selecting select ultrasound data along the first 2D plane when the anatomical structure is symmetric.

In an embodiment a tangible and non-transitory computer readable medium comprising one or more programmed instructions is provided. The one or more programmed instructions are configured to direct one or more processors. The one or more processors may be directed to acquire ultrasound data along first and second two dimensional (2D) planes from a matrix array probe. The second 2D plane includes an anatomical structure. The first 2D plane extending along the azimuth direction and the second 2D plane extending along the elevation direction. The one or more processor may further be directed to identify when the anatomical structure is symmetric along the second 2D plane with respect to a characteristic of interest, and select select ultrasound data along the first 2D plane when the anatomical structure is symmetric.

### BRIEF DESCRIPTION OF THE DRAWINGS

FIG. 1 is an illustration of a schematic block diagram of an ultrasound imaging system, in accordance with an embodiment.

FIG. 2A is an illustration of an ultrasound probe of an embodiment along an azimuth plane of the ultrasound imaging system shown in FIG. 1.

FIG. 2B is an illustration of an ultrasound probe of an embodiment along an elevation plane of the ultrasound imaging system shown in FIG. 1.

FIG. 3 is an illustration of two dimensional planes of an ultrasound probe of an embodiment of the ultrasound imaging system shown in FIG. 1.

FIG. 4 is an illustration of an adjustment of a position of a two dimensional plane of an embodiment of the ultrasound imaging system shown in FIG. 1.

FIGS. 5A-B are illustrations of ultrasound images of an embodiment along a two dimensional plane.

FIG. 6 is a flow chart of a method in accordance with an embodiment.

FIG. 7 is an illustration of ultrasound images along two dimensional planes, in accordance with embodiments described herein.

FIG. 8 is an illustration of ultrasound images along two dimensional planes, in accordance with embodiments described herein.

### DETAILED DESCRIPTION OF THE INVENTION

The following detailed description of certain embodiments will be better understood when read in conjunction with the appended drawings. To the extent that the figures illustrate diagrams of the functional modules of various embodiments, the functional blocks are not necessarily indicative of the division between hardware circuitry. Thus, for example, one or more of the functional blocks (e.g., processors or memories) may be implemented in a single piece of hardware (e.g., a general purpose signal processor or a block of random access memory, hard disk, or the like). Similarly, the programs may be stand-alone programs, may be incorporated as subroutines in an operating system, may be functions in an installed software package, and the like. It should be understood that the various embodiments are not limited to the arrangements and instrumentality shown in the drawings.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising” or “having” an element or a plurality of elements having a particular property may include additional elements not having that property.

Various embodiments provide systems and methods for selecting a two dimensional (2D) scan plane using a medical diagnostic imaging system, such as an ultrasound imaging system. The select 2D scan plane (e.g., mid-sagittal plane) is selected based on identifying symmetry of anatomical structures along a perpendicular plane relative to the select 2D scan plane. The symmetry of the anatomical structures may be identified based on machine learning algorithms. For example, the ultrasound imaging system is configured to acquire ultrasound data along two orthogonal planes, a first plane representing the select 2D scan plane and a second plane orthogonal to the select 2D scan plane. A position of the ultrasound probe may be intermittently and/or continually adjusted by the user during the scan. As ultrasound data is acquired, the ultrasound imaging system is configured to analyze the ultrasound data along the second plane. For example, the ultrasound imaging system is configured to identify when one or more anatomical structures along the second plane are symmetric. When the one or more anatomical structures are symmetric, the ultrasound imaging system is configured to notify the user and/or select the ultrasound data along the select 2D plane.

At least one technical effect of various embodiments described herein provide increasing the accuracy of finding a 2D scan plane. At least one technical effect of various embodiments described herein reduces a scan time of a medical diagnostic imaging system.

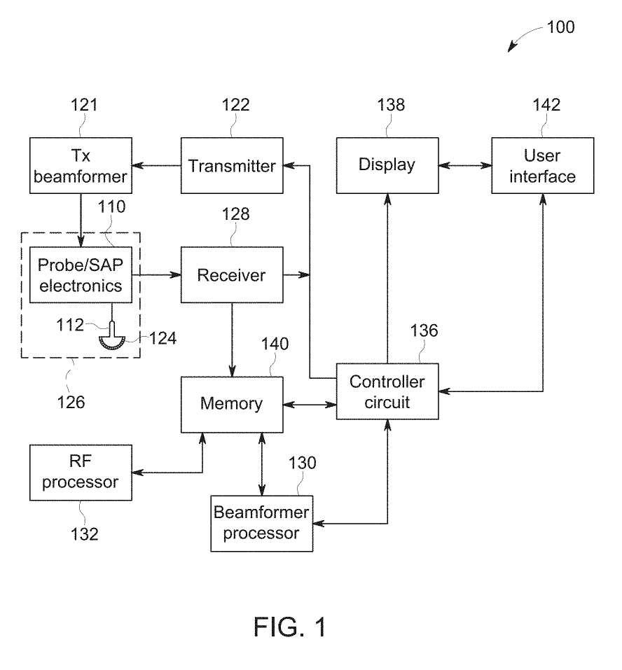

FIG. 1 is a schematic diagram of a diagnostic medical imaging system, specifically, an ultrasound imaging system 100. The ultrasound imaging system 100 includes an ultrasound probe 126 having a transmitter 122, transmit beamformer 121 and probe/SAP electronics 110. The probe/SAP electronics 110 may be used to control the switching of the transducer elements 124. The probe/SAP electronics 110 may also be used to group transducer elements 124 into one or more sub-apertures.

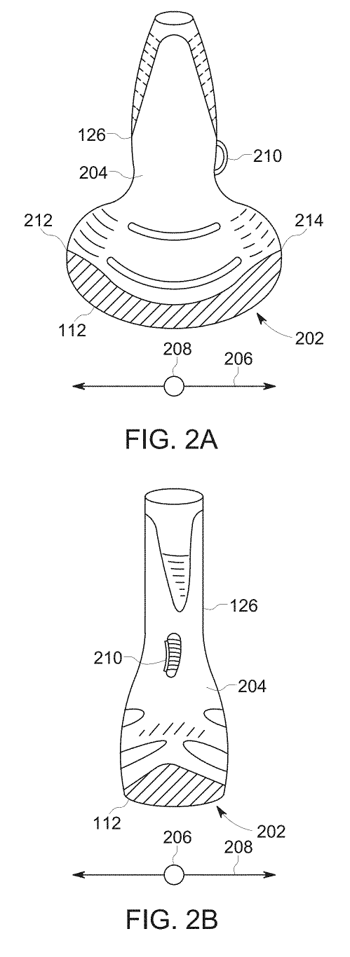

The ultrasound probe 126 may be configured to acquire ultrasound data or information from a region of interest (ROI) (e.g., organ, blood vessel, heart, brain, fetal tissue, cardiovascular, neonatal brain, embryo, abdomen, and/or the like) that includes one or more anatomical structures of the patient. The ultrasound probe 126 is communicatively coupled to the controller circuit 136 via the transmitter 122. The transmitter 122 transmits a signal to a transmit beamformer 121 based on acquisition settings received by the controller circuit 136. The acquisition settings may define an amplitude, pulse width, frequency, and/or the like of the ultrasonic pulses emitted by the transducer elements 124. The transducer elements 124 emit pulsed ultrasonic signals into a patient (e.g., a body). The acquisition settings may be adjusted by the user by selecting a gain setting, power, time gain compensation (TGC), resolution, and/or the like from the user interface 142. The signal transmitted by the transmitter 122 in turn drives a plurality of transducer elements 124 within a transducer array 112. In connection with FIGS. 2A-B, the transducer array 112 may be a matrix array of transducer elements 124 arranged to include an elevation direction and an azimuth direction. For example only, the transducer array 112 may include an array of 128 transducer elements 124 along the azimuth plane 206 and along the elevation plane 208 to from a matrix array probe (e.g., the ultrasound probe 126).

FIG. 2A illustrates the ultrasound probe 126 of an embodiment along an azimuth plane 206. The ultrasound probe 126 includes a housing 204 configured to enclose the probe/SAP electronics 110 and affix the transducer array 112 to a front end 202 of the ultrasound probe 126. The housing 204 may include one or more user interface components 210, such as a tactile button, rotary button, capacitive button, and/or the like. The front end 202 of the housing 204 shown in FIG. 2A is configured to hold and/or confine the transducer array 112, which is shown extending along the azimuth plane 206, to the housing 202. The azimuth plane 206 is shown as a standard plane extending along a length of the ultrasound probe 126. It may be noted a variety of a geometries and/or configurations may be used for the transducer array 112. For example, the transducer elements 124 of the transducer array 112 forms a curved surface area of the ultrasound probe 126 such that opposing ends 212, 214 of the transducer array 112 deviates from a center portion of the transducer array 112.

FIG. 2B illustrates the ultrasound probe 126 of an embodiment along an elevation plane 208. The elevation plane 208 is orthogonal to the azimuth plane 206. For example, the ultrasound probe 126 shown in FIG. 2B is a side view relative to the ultrasound probe 126 of FIG. 2A.

Returning to FIG. 1, the transducer elements 124 emit pulsed ultrasonic signals into a body (e.g., patient) or volume corresponding to the acquisition settings along one or more scan planes. The ultrasonic signals may include, for example, one or more reference pulses, one or more pushing pulses (e.g., shear-waves), and/or one or more pulsed wave Doppler pulses. At least a portion of the pulsed ultrasonic signals back-scatter from the ROI (e.g., heart, left ventricular outflow tract, breast tissues, liver tissues, cardiac tissues, prostate tissues, neonatal brain, embryo, abdomen, and/or the like) to produce echoes. The echoes are delayed in time and/or frequency according to a depth or movement, and are received by the transducer elements 124 within the transducer array 112. The ultrasonic signals may be used for imaging, for generating and/or tracking shear-waves, for measuring changes in position or velocity within the ROI (e.g., flow velocity, movement of blood cells), differences in compression displacement of the tissue (e.g., strain), and/or for therapy, among other uses. For example, the probe 126 may deliver low energy pulses during imaging and tracking, medium to high energy pulses to generate shear-waves, and high energy pulses during therapy.

The transducer elements 124 convert the received echo signals into electrical signals which may be received by a receiver 128. The receiver 128 may include one or more amplifiers, an analog to digital converter (ADC), and/or the like. The receiver 128 may be configured to amplify the received echo signals after proper gain compensation and convert these received analog signals from each transducer element 124 to digitized signals sampled uniformly in time. The digitized signals representing the received echoes are stored on memory 140, temporarily. The digitized signals correspond to the backscattered waves received by each transducer element 124 at various times. After digitization, the signals still may preserve the amplitude, frequency, phase information of the backscatter waves.

Optionally, the controller circuit 136 may retrieve the digitized signals stored in the memory 140 to prepare for the beamformer processor 130. For example, the controller circuit 136 may convert the digitized signals to baseband signals or compressing the digitized signals.

The beamformer processor 130 may include one or more processors. Optionally, the beamformer processor 130 may include a central controller circuit (CPU), one or more microprocessors, or any other electronic component capable of processing inputted data according to specific logical instructions. Additionally or alternatively, the beamformer processor 130 may execute instructions stored on a tangible and non-transitory computer readable medium (e.g., the memory 140) for beamforming calculations using any suitable beamforming method such as adaptive beamforming, synthetic transmit focus, aberration correction, synthetic aperture, clutter reduction and/or adaptive noise control, and/or the like. Optionally, the beamformer processor 130 may be integrated with and/or apart of the controller circuit 136. For example, the operations described being performed by the beamformer processor 130 may be configured to be performed by the controller circuit 136.

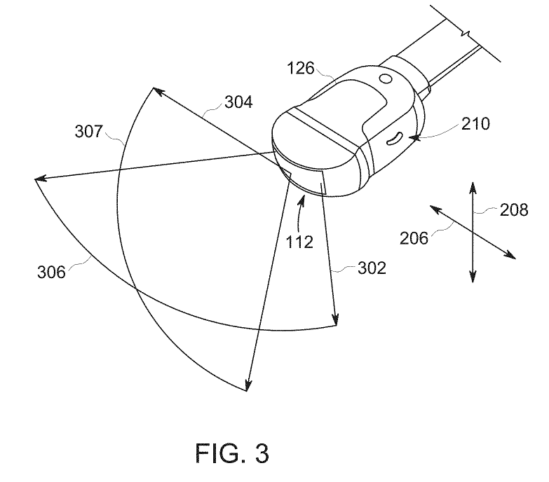

In connection with FIG. 3, the beamformer processor 130 may be configured to acquire ultrasound data concurrently along two 2D planes 302, 304.

FIG. 3 is an illustration of the 2D planes 302, 304 of the ultrasound probe 126 of an embodiment of the ultrasound imaging system 100. The 2D planes 302, 304 may each define a 2D area extending from the transducer array 112 of the ultrasound imaging system 100 that acquires ultrasound data. The 2D planes 302, 304 are orthogonal with respect to each other. For example, the 2D plane 302 extends along the azimuth direction (e.g., parallel to the azimuth plane 206), and the 2D plane 304 extends along the elevation direction (e.g., parallel to the elevation plane 208).

During a bi-plane imaging mode of the ultrasound imaging system 100, the beamformer processor 130 is configured to beamform ultrasound data along the 2D planes 302, 304. For example, the beamformer processors 130 may be configured to define the 2D planes 302, 304. Based on the 2D planes 302, 304 the beamformer processor 130 may be configured to perform filtering and/or decimation, to isolate and/or select the digitized signals corresponding to select transducer elements 124 of the transducer array 112 along the 2D planes 302, 304. The select transducer elements 124 represent active footprints selected for beamforming that define the 2D planes 302 and 304. The beamformer processor 130 may define channels and/or time slots of the digitized data that correspond to the selected transducer elements 124 that may be beamformed, with the remaining channels or time slots of digitized data (e.g., representing transducer elements 124 not within the active footprints representing the 2D planes 302, 304) that may not be communicated for processing (e.g., discarded). It may be noted that the ultrasound data corresponding to the area along the 2D planes 302 and 304 may be acquired concurrently and/or simultaneously by the ultrasound probe 126. Additionally or alternatively, the beamformer processor 130 is configured to process the digitized data corresponding to the transducer elements 124 defining the 2D planes 302 and 304 concurrently and/or simultaneously.

Each of the 2D planes 302 and 304 extend along the azimuth plane 206 and the elevation plane 208 defining imaging angles 306, 306. For example, the imaging angle 306 of the 2D plane 302 extends along the azimuth direction, and the imaging angle 307 of the 2D plane 304 extends along the elevation direction. The imaging angles 306, 307 may correspond to a 2D sweep angle centered at a virtual apex defining a range along the azimuth and elevation planes 206, 208 from the transducer array 112 the controller circuit 136 is configured to acquire ultrasound data. A size (e.g., length along the azimuth direction, length along the elevation direction) of the imaging angles 302, 304 may be adjusted by the beamformer processor 130 and/or the controller circuit 136. For example, the size of the imaging angle 307 of the 2D plane 304 may correspond to an array of select transducer elements 124 along the elevation plane 208 to define the length of the imaging angle 307 selected by the beamformer processor 130. In another example, the controller circuit 136 may instruct the beamformer processor 130 to adjust the length based on instructions received from the user interface component 210 and/or a user interface 142. The controller circuit 136 may be configured to adjust a size of the imaging angle 306 by adjusting a number of transducer elements 124 along the azimuth plane 206 included in the digitized signals by the beamformer processor 130. In another example, the controller circuit 136 may be configured to adjust a size of the imaging angle 307 by adjusting a number of transducer elements 124 along the elevation plane 208 included in the digitized signals by the beamformer processor 130.

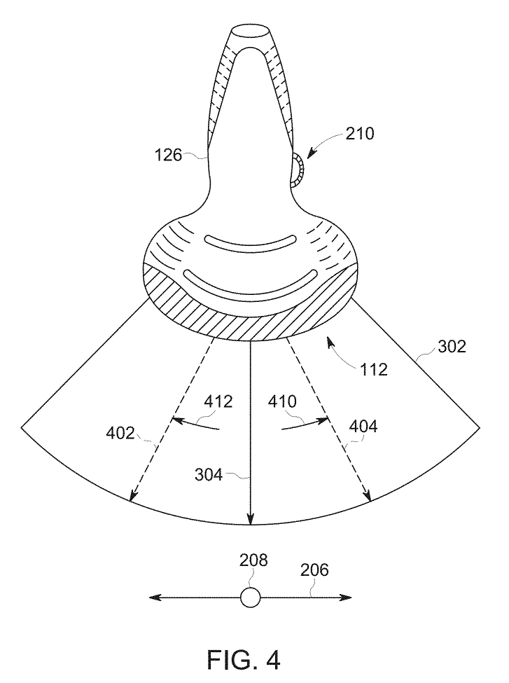

The 2D plane 304 shown in FIG. 3, is shown at a mid-position and/or zero degree position of the 2D plane 302. In connection with FIG. 4, the controller circuit 136 may be configured to adjust a position of the 2D plane 304 along the azimuth direction and/or with respect to the 2D plane 302.

FIG. 4 is an illustration of an adjustment of a position of the two dimensional plane 304 of an embodiment of the ultrasound imaging system 100. For example, the illustration shown in FIG. 4 is shown along the azimuth plane 206 of the ultrasound probe 126. The controller circuit 136 may adjust the select transducer elements 124 corresponding to the 2D plane 304 along the azimuth direction in a direction of arrows 410 or 412.

For example, the controller circuit 136 may receive instruction from the user interface component 210 and/or the user interface 142 to shift the 2D plane 304 in the direction of the arrow 412. Based on the instruction, the controller circuit 136 may instruct the beamformer processor 130 to select an alternative selection of the transducer elements 124 along the transducer array 112 in the direction of the arrow 412. The alternative selection of transducer elements 124 utilized by the beamformer processor 130 may form an alternative 2D plane 402 aligned along the elevation direction.

In another example, the controller circuit 136 may receive instruction from the user interface component 210 and/or the user interface 142 to shift the 2D plane 304 in the direction of the arrow 410. Based on the instruction, the controller circuit 136 may instruct the beamformer processor 130 to select an alternative selection of the transducer elements 124 along the transducer array 112 in the direction of the arrow 410. The alternative selection of transducer elements 124 utilized by the beamformer processor 130 may form an alternative 2D plane 404 aligned along the elevation direction.

Returning to FIG. 1, the beamformer processor 130 performs beamforming on the digitized signals of transducer elements 124 corresponding to the 2D planes 302 and 304, and outputs a radio frequency (RF) signal. The RF signal is then provided to an RF processor 132 that processes the RF signal. The RF processor 132 may include one or more processors. Optionally, the RF processor 132 may include a central controller circuit (CPU), one or more microprocessors, or any other electronic component capable of processing inputted data according to specific logical instructions. Additionally or alternatively, the RF processor 132 may execute instructions stored on a tangible and non-transitory computer readable medium (e.g., the memory 140). Optionally, the RF processor 132 may be integrated with and/or apart of the controller circuit 136. For example, the operations described being performed by the RF processor 132 may be configured to be performed by the controller circuit 136.

The RF processor 132 may generate different ultrasound image data types, e.g. B-mode, color Doppler (velocity/power/variance), tissue Doppler (velocity), and Doppler energy, for multiple scan planes or different scanning patterns. For example, the RF processor 132 may generate tissue Doppler data for multi-scan planes. The RF processor 132 gathers the information (e.g. I/Q, B-mode, color Doppler, tissue Doppler, and Doppler energy information) related to multiple data slices and stores the data information, which may include time stamp and orientation/rotation information, in the memory 140.

Alternatively, the RF processor 132 may include a complex demodulator (not shown) that demodulates the RF signal to form IQ data pairs representative of the echo signals. The RF or IQ signal data may then be provided directly to the memory 140 for storage (e.g., temporary storage). Optionally, the output of the beamformer processor 130 may be passed directly to the controller circuit 136.

The controller circuit 136 may be configured to process the acquired ultrasound data (e.g., RF signal data or IQ data pairs) and prepare and/or generate frames of ultrasound image data representing an ultrasound image of the ROI for display on the display 138. The ultrasound image data may represent on the ultrasound data acquired along one and/or both of the 2D planes 302 and 304. For example, the controller circuit 136 may display an ultrasound image of the ROI along the 2D plane 302 and/or the 2D plane 304 on the display 138. Additionally or alternatively, the controller circuit 136 may display ultrasound images of both the 2D planes 302 and 304 concurrently and/or simultaneously on the display 138.

The controller circuit 136 may include one or more processors. Optionally, the controller circuit 136 may include a central controller circuit (CPU), one or more microprocessors, a graphics controller circuit (GPU), or any other electronic component capable of processing inputted data according to specific logical instructions. Having the controller circuit 136 that includes a GPU may be advantageous for computation-intensive operations, such as volume-rendering. Additionally or alternatively, the controller circuit 136 may execute instructions stored on a tangible and non-transitory computer readable medium (e.g., the memory 140).

The controller circuit 136 is configured to perform one or more processing operations according to a plurality of selectable ultrasound modalities on the acquired ultrasound data, adjust or define the ultrasonic pulses emitted from the transducer elements 124, adjust one or more image display settings of components (e.g., ultrasound images, interface components, positioning regions of interest) displayed on the display 138, and other operations as described herein. Acquired ultrasound data may be processed in real-time by the controller circuit 136 during a scanning or therapy session as the echo signals are received. Additionally or alternatively, the ultrasound data may be stored temporarily in the memory 140 during a scanning session and processed in less than real-time in a live or off-line operation.

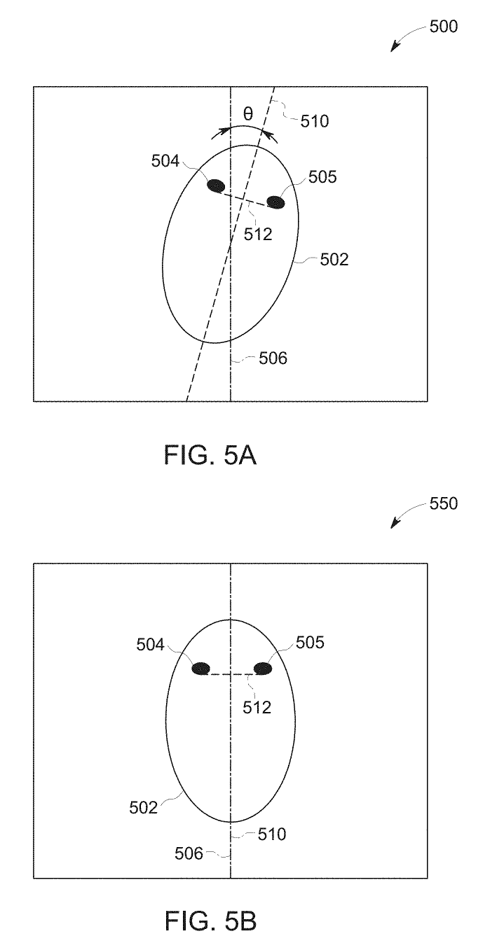

The controller circuit 136 is configured to identify when an anatomical structure (e.g., anatomical structure 502, 504, 505 of FIG. 5) of the 2D plane 304 is symmetric with respect to a characteristic of interest.

In at least one embodiment, the characteristic of interest may represent orientation, angle, form, and/or the like of a plurality of subsets of the shape of the anatomical structure 502. The subsets may represent equally subdivided portions of the anatomical structure 502. The symmetry of the anatomical structure 502 may occur when at least two of the subsets are a reflection of each other about a symmetrical axis 510. For example, the controller circuit 136 may determine the symmetrical axis 510 representing the symmetry of the anatomical structure based on a shape of the anatomical structure and/or based on a position of the anatomical structure relative to one or more alternative anatomical structures. Based on an orientation of the symmetrical axis 510 the controller circuit 136 may determine when the anatomical structure of the 2D plane 304 is symmetrically aligned with the 2D plane 302.

FIGS. 5A-B are illustrations of ultrasound images 500 and 550 of an embodiment along the 2D plane 304. The ultrasound images 500 and 550 include the anatomical structure 502 within the ROI of the ultrasound imaging system 100. For example, the anatomical structure 502 may represent a bone structure (e.g., skull, femur, pelvis, and/or the like), organ (e.g., heart, bladder, kidney, liver, and/or the like), uterus, and/or the like. The ultrasound images 500 and 550 may represent different positions of the 2D plane 304 within the patient. For example, during the scan the user may intermittently and/or continuously re-position the ultrasound probe 126 with respect to the patient resulting in the separate ultrasound images 500 and 550. In another example, the controller circuit 136 may adjust a position of the 2D plane 304, as described in connection with FIG. 4, based on instructions received from the user interface component 210 and/or the user interface 142.

The controller circuit 136 may determine the symmetry of a shape of the anatomical structure 502 by executing a machine learning algorithm stored in the memory 140. For example, the machine learning algorithm may represent a model based on decision tree learning, neural network, deep learning, representation learning, and/or the like. The model may be configured to determine a symmetrical axis 510 based on the overall shape of the anatomical structure 502.

The shape of the anatomical structure 502 may be determined based on an edge detection. For example, the controller circuit 136 may determine edges of the anatomical structure 502 based on one or more feature vectors determined from each pixel of the ultrasound image 500. One of the feature vectors sets may be based on an intensity histogram of the ultrasound image 500. In another example, when executing the model the controller circuit 136 may calculate feature vectors based on a mean intensity of the plurality of pixels, a variance of the plurality of pixel intensities, a kurtosis or shape of intensity distribution of the plurality of pixels, a skewness of the plurality of pixels, and/or the like. Based on changed in the feature vectors between the pixels, the controller circuit 136 may identify a boundary of the anatomical structure 502. Optionally, the model may include a k-means clustering and/or random forest classification to define the feature vectors corresponding to the boundary of the pixels. The feature vectors represent characteristics of the pixels and/or adjacent pixels which are utilized to locate the boundary of the anatomical structure 502. Optionally, the model may be generated and/or defined by the controller circuit 136 based from a plurality of reference ultrasound images.

Additionally or alternative, the controller circuit 136 may be configured to detect the anatomical structure 502 by applying thresholding or border detection methods to identity objects having a particular shape or size, which may be based on, for example, a type of examination or a user input of the anatomy scanned by the ultrasound imaging system 100. For example, in the case of a fetal biometry scan of the head, the controller circuit 136 may search for a circular structure within the ultrasound image 500. Additionally or alternatively, the controller circuit 136 may utilize a pattern recognition technique, a machine learning algorithm, correlation, statistical analysis or linear regression approach may be used to identify the anatomical structure 502.

Based on the boundary of the anatomical structure 502, the controller circuit 136 may determine a shape of the anatomical structure 502. The shape may be utilized by the controller circuit 136 to determine the symmetrical axis 510 of the anatomical structure 502. The symmetrical axis 510 may represent an approximate reflection symmetry of the anatomical structure 502. For example, the symmetrical axis 510 may be interposed within the anatomical structure 502 defining opposing ends of the boundary of the anatomical structure 502. A position of the symmetrical axis 510 may be configured such that the opposing ends are an approximate reflection of each other about the symmetrical axis 510.

Additionally or alternatively, the controller circuit 136 may determine the symmetry of the anatomical structure 504 based on a position of the anatomical structure 504 with respect to a second anatomical structure 505. For example, the anatomical structures 504 and 505 may represent a pair of like organs (e.g., kidney, lungs, ovary), cavity (e.g., orbit), nerve structure (e.g., olfactory, optical nerve, trigeminal), bone structure, and/or the like. The characteristic of interest may represent a relative position, distance, orientation, and/or the like between two different anatomical structures 504, 505. The controller circuit 136 may determine positions of the anatomical structures 504 and 505 by executing the machine learning algorithm stored in the memory 140. For example, the controller circuit 136 may execute a model defined by the machine learning algorithm (e.g., decision tree learning, neural network, deep learning, representation learning, and/or the like). The controller circuit 136 may compare an intensity or brightness of the pixels of the ultrasound image 500 to feature vectors of the model. In another example, the controller circuit 136 may determine a variance kurtosis, skewness, or spatial distribution characteristic of the select pixel by comparing the intensity of the select pixel with adjacent and/or proximate pixels to identify the anatomical structures 504 and 505.

Each feature vector may be an n-dimensional vector that includes three or more features of pixels (e.g., mean, variance, kurtosis, skewness, spatial distribution) corresponding to the pixels representing the anatomical structures 504 and 505 within the ultrasound image 500. The feature vectors of the model may be generated and/or defined by the controller circuit 136 based from a plurality of reference ultrasound images that include the anatomical structures 504 and 505. For example, the controller circuit 136 may select pixel blocks from one hundred reference ultrasound images. The select pixel blocks may have a length of five pixels and a width of five pixels. The select pixel blocks may be selected and/or marked by the user to correspond to the anatomical structures 504 and 505. For example, a plurality of pixels within each select pixel block may represent and/or correspond to one of the anatomical structures 504 and 505. Based on the plurality of pixels within the select pixel blocks, the controller circuit 136 may generate and/or define a feature vector of the model configured to identify the anatomical structures 504 and 505.

Based on the identified position of the anatomical structures 504 and 505 by the controller circuit 136, the controller circuit 136 may determine a positional axis 512. The positional axis 512 may represent the relative positions of the anatomical structures 504 and 505. Based on the positional axis 512 the controller circuit 136 may determine the symmetrical axis 510. For example, based on the lateral position of the anatomical structures 504 and 505, the controller circuit 136 may determine that the symmetrical axis 510 is perpendicular to the positional axis 512.

The controller circuit 136 may determine when the anatomical structure 502 is symmetric based on an orientation of the symmetrical axis 510 relative to the 2D plane 302. For example, the 2D plane 302 is perpendicular to the ultrasound images 500 and 550 and is represented at the axis 506. The controller circuit 136 may compare the orientation and/or position of the symmetrical axis 510 with the axis 506. For example, the controller circuit 136 may determine that the symmetrical axis 510 is shifted with respect to the axis 506 at an angle, θ. Based on the difference in orientation, the controller circuit 136 may determine that the anatomical structure 502 is not symmetric with the 2D plane 302.

Optionally, the controller circuit 136 may display a notification on the display 138 to adjust the position of the 2D plane 304 within the patient. For example, the notification may be a pop-up window, a graphical icon, graphical flashes, textual information and/or the like configured to indicate to the user to adjust a position of the ultrasound probe 126 and/or the 2D plane 304. Additionally or alternatively, the notification may be an auditory alert.

In connection with the ultrasound image 550, the controller circuit 136 may determine that the anatomical structure 502 is symmetrical with respect to the 2D plane 302. For example, the controller circuit 136 may compare the orientation of the symmetrical axis 510 of the ultrasound image with the axis 506. When a difference in orientation is below a predetermined threshold (e.g., less than one degree), the controller circuit 136 may determine that the symmetrical axis 510 of the ultrasound image 550 is aligned with the axis 506. Based on the determination of the alignment of the symmetrical axis 510 and the axis 506, the controller circuit 136 is configured to determine that the anatomical structure 502 is aligned with the 2D plane 302. Optionally, the controller circuit 136 may display a notification on the display 138 that the 2D plane 304 is in symmetry with the 2D plane 302. For example, the notification may be a pop-up window, a graphical icon, graphical flashes, textual information and/or the like configured to indicate that the 2D plane 302 is correctly aligned. Additionally or alternatively, the notification may be an auditory alert.

Returning to FIG. 1, the memory 140 may be used for storing processed frames of acquired ultrasound data that are not scheduled to be displayed immediately or to store post-processed images (e.g., shear-wave images, strain images), firmware or software corresponding to, for example, the machine learning algorithms, a graphical user interface, one or more default image display settings, programmed instructions (e.g., for the controller circuit 136, the beamformer processor 130, the RF processor 132), and/or the like. The memory 140 may be a tangible and non-transitory computer readable medium such as flash memory, RAM, ROM, EEPROM, and/or the like.

The controller circuit 136 is operably coupled to the display 138 and the user interface 142. The display 138 may include one or more liquid crystal displays (e.g., light emitting diode (LED) backlight), organic light emitting diode (OLED) displays, plasma displays, CRT displays, and/or the like. The display 138 may display patient information, ultrasound images and/or videos, components of a display interface, one or more ultrasound images generated from the ultrasound data stored in the memory 140 or currently being acquired, measurements, diagnosis, treatment information, and/or the like received by the display 138 from the controller circuit 136.

The user interface 142 controls operations of the controller circuit 136 and is configured to receive inputs from the user. The user interface 142 may include a keyboard, a mouse, a touchpad, one or more physical buttons, and/or the like. Based on selections received by the user interface 142 the controller circuit 136 may adjust the position of the 2D plane 304, the imaging angles 306 and 307 of the 2D planes 302 and 304, and/or the like. Optionally, the display 138 may be a touchscreen display, which includes at least a portion of the user interface 142.

For example, a portion of the user interface 142 shown on a touchscreen display (e.g., the display 138) is configured to receive one or more selections associated and/or represented as a graphical user interface (GUI) generated by the controller circuit 136 shown on the display. The GUI may include one or more interface components that may be selected, manipulated, and/or activated by the user operating the user interface 142 (e.g., touchscreen, keyboard, mouse). For example, the controller circuit 136 is configured to adjust a position of the 2D plane 304 based on the selection of the one or more interface components of the GUI. The interface components may be presented in varying shapes and colors, such as a graphical or selectable icon, a slide bar, a cursor, and/or the like. For example, one of the interface components shown on the GUI may be a notification to adjust the ultrasound probe 126 and/or the 2D plane 304. In another example, one of the interface components shown on the GUI may be a notification that the 2D plane 302 is aligned, such as representing a mid-sagittal view of the patient. Optionally, one or more interface components may include text or symbols, such as a drop-down menu, a toolbar, a menu bar, a title bar, a window (e.g., a pop-up window) and/or the like. Additionally or alternatively, one or more interface components may indicate areas within the GUI for entering or editing information (e.g., patient information, user information, diagnostic information), such as a text box, a text field, and/or the like.

In various embodiments, the interface components may perform various functions when selected, such as adjusting (e.g., increasing, decreasing) one or both of the imaging angles 306, 307, adjusting a position of the 2D plane 304 along the azimuth direction, selecting the scan being performed by the ultrasound imaging system 100, measurement functions, editing functions, database access/search functions, diagnostic functions, controlling acquisition settings, and/or system settings for the ultrasound imaging system 100 performed by the controller circuit 136.

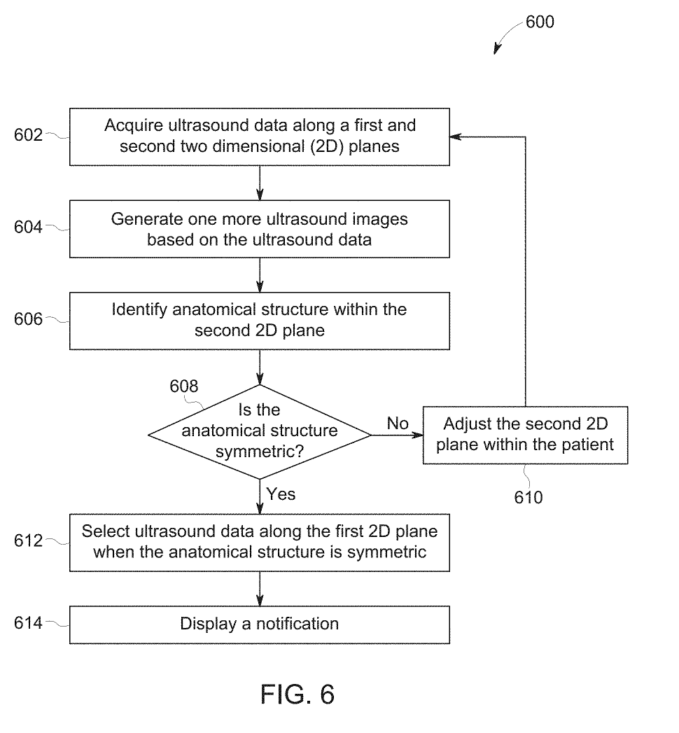

FIG. 6 is a flow chart of a method 600 in accordance with an embodiment. The method 600 may be, for example, selecting a two dimensional (2D) scan plane during a scan of the ultrasound imaging system 100. The method 600 may employ structures or aspects of various embodiments (e.g., the controller circuit 136, the ultrasound probe 126, the ultrasound imaging system 100, and/or the like) discussed herein. In various embodiments, certain steps may be omitted or added, certain steps may be combined, certain steps may be performed simultaneously, certain steps may be performed concurrently, certain steps may be split into multiple steps, certain steps may be performed in a different order, or certain steps or series of steps may be re-performed in an iterative fashion.

Beginning at 602, the controller circuit 136 may be configured to acquire ultrasound data along a first and second 2D plane. For example, the controller circuit 136 may instruct the beamformer processor 130 to select digitized signals received from the ultrasound probe 126 corresponding to the 2D planes 302, 304 (FIG. 3). The select digitized signals may correspond to transducer elements aligned along the azimuth plane 206 and elevation plane 208 representing the 2D plane 302 and 304, respectively. For example, the beamformer processor 130 may be configured to perform filtering and/or decimation, to isolate and/or select the digitized signals corresponding to the relevant transducer elements 124 of the transducer array 112 along the 2D planes 302, 304 representing active footprints selected for beamforming. The digitized signals are beamformed by the beamformer processor 130, and output the RF signal processed to the RF processor 132. The processed RF signals are stored as ultrasound data in the memory 140, which is acquired by the controller circuit 136.

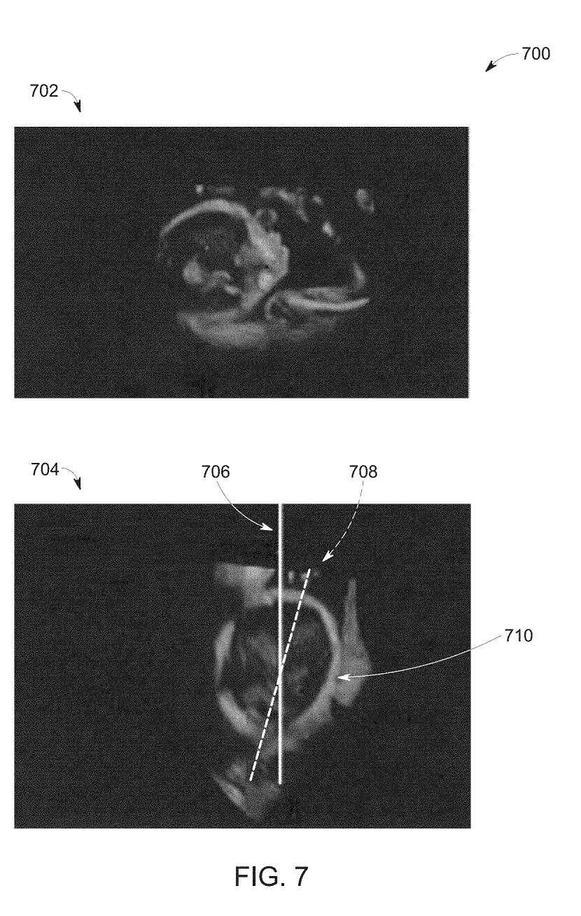

At 604, the controller circuit 136 may be configured to generate one or more ultrasound images based on the ultrasound data. The one or more ultrasound images may be displayed on the display 138 during the acquisition of the ultrasound data. In connection with FIG. 7, the one or more ultrasound images 702, 704 may represent the ultrasound data acquired along the 2D planes 302 and 304.

FIG. 7 is an illustration 700 of the ultrasound images 702, 704 along the 2D planes 302, 304, in accordance with embodiments described herein. The ultrasound image 702 represents the 2D plane 302, and the 2D ultrasound image 704 represents the 2D plane 304. The ultrasound images 702 and 704 may be displayed concurrently and/or simultaneously on the display 138. Additionally or alternatively, the controller circuit 136 may display one of the ultrasound images 702, 704 based on instructions received from the user interface 142.

At 606, the controller circuit 136 may be configured to identify an anatomical structure 710 within the second 2D plane. The controller circuit 136 may identify the anatomical structure 710 by applying segmentation and/or border detection methods. For example, the controller circuit 136 may be configured to detect the anatomical structure 710 by applying thresholding or border detection methods to identity objects having a particular shape or size, which may be based on, for example, a type of examination or a user input of the anatomy scanned by the ultrasound imaging system 100. For example, in the case of a fetal biometry scan of the head, the controller circuit 136 may search for a circular structure within the ultrasound image 704. Additionally or alternatively, the controller circuit 136 may utilize a pattern recognition technique, a machine learning algorithm, correlation, statistical analysis or linear regression approach may be used to identify the anatomical structure 710.

At 608, the controller circuit 136 may be configured to determine when the anatomical structure of the second 2D plane is symmetric. For example, the controller circuit 136 may determine the symmetry of a shape of the anatomical structure 710 by executing the model defined by the machine learning algorithm stored in the memory 140. Based on the boundary of the anatomical structure 710, the model executed by the controller circuit 136 may define a symmetrical axis 708. The symmetrical axis 708 may represent an approximate reflection symmetry of the anatomical structure 710. For example, the symmetrical axis 708 may be interposed within the anatomical structure 710 defining opposing ends of the boundary of the anatomical structure 710. A position of the symmetrical axis 708 may be configured such that the opposing ends are an approximate reflection of each other about the symmetrical axis 710.

The controller circuit 136 may determine when the anatomical structure 710 is symmetric based on an orientation of the symmetrical axis 708 relative to the 2D plane 302 represented as the axis 706. The controller circuit 136 may compare the orientation and/or position of the symmetrical axis 708 with the axis 706. For example, the controller circuit 136 may determine that the symmetrical axis 708 is shifted with respect to the axis 706. Based on the difference in orientation between the axes 706 and 708, the controller circuit 136 may determine that the anatomical structure 502 is not symmetric with the 2D plane 302.

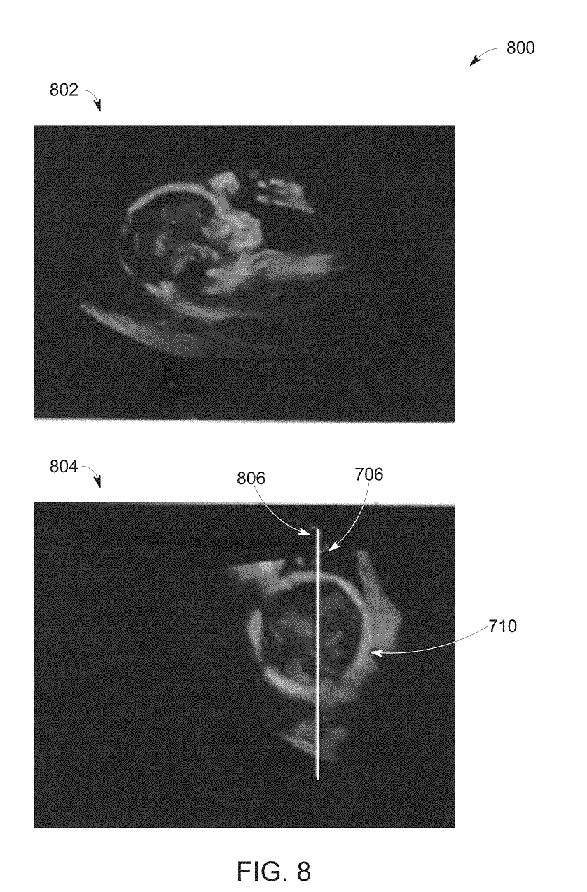

If the anatomical structure is not symmetric, then at 610, the controller circuit 136 may be configured to adjust the second 2D plane within the patient. For example, the controller circuit 136 may display a notification on the display 138. The notification may be an interface component shown on the GUI configured to notify the user based on textual information, graphical icon, animation, set color, and/or the like to adjust the ultrasound probe 126 and/or the 2D plane 304. Optionally, the controller circuit 136 may continually acquire ultrasound data along the first and second 2D plane while the ultrasound probe 126 and/or the 2D plane 304 is adjusted by the user. For example, the controller circuit 136 may acquire additional ultrasound data based on the adjustment by the user of the ultrasound probe 126 and/or the 2D plane 304. In connection with FIG. 8, the additional ultrasound data is represented by the ultrasound images 802 and 804.

FIG. 8 is an illustration 800 of the ultrasound images 802, 804 along the 2D planes 302, 304, in accordance with embodiments described herein. The ultrasound image 802 represents the 2D plane 302, and the 2D ultrasound image 804 represents the 2D plane 304. The anatomical structure 710 shown in the ultrasound image 804 is adjusted with respect to the anatomical structure 710 shown in the ultrasound image 704. Based on the adjustment of the anatomical structure 710 of the ultrasound image 804, the controller circuit 136 may determine a new symmetrical axis 806. For example, the controller circuit 136 may determine the symmetry of a shape of the anatomical structure 710 by executing the model defined by the machine learning algorithm stored in the memory 140. Based on the boundary of the anatomical structure 710, the model executed by the controller circuit 136 may define the symmetrical axis 806. The controller circuit 136 may determine that the anatomical structure 710 shown in the ultrasound image 804 is symmetric based on an orientation of the symmetrical axis 806 relative to the 2D plane 302 represented as the axis 706. For example, the controller circuit 136 may compare the orientation and/or position of the symmetrical axis 806 with the axis 706, which is shown in the ultrasound image 804 being aligned with each other.

If the anatomical structure is symmetric, then at 612, the controller circuit 136 may be configured to select ultrasound data along the first 2D plane. For example, the first 2D plane may be automatically selected by the controller circuit 136 at a line of the symmetrical axis 806 through the second 2D plane. The select ultrasound data represents ultrasound data acquired along the first 2D plane (e.g., the 2D plane 302) acquired concurrently and/or simultaneously when the second 2D plane is determined by the controller circuit 136 to be symmetric. For example, the ultrasound data acquired along the 2D planes 302 and 304 are acquired concurrently and/or simultaneously representing the ultrasound images 802 and 804, respectively. The controller circuit 136 is configured to determine that the 2D plane 304 is symmetric based on the alignment between the symmetrical axis 806 with the axis 706. Based on the determination by the controller circuit 136 the 2D plane 304 is symmetric, the controller circuit 136 is configured to select the ultrasound data represented by the ultrasound image 802. For example, the controller circuit 136 is configured to select ultrasound data acquired along the 2D plane 302 that was concurrently and/or simultaneously acquired with the ultrasound data along the 2D plane 304 that is symmetric.

At 614, the controller circuit 136 may be configured to generate a notification. The notification may be configured to inform the user that the 2D scan plane (e.g., mid-sagittal plan) of the patient has been acquired. For example, the controller circuit 136 is configured to generate a pop-up window, animation, a graphical icon, and/or the like on the display 138. Additionally or alternatively, the notification may be an interface component. For example, the controller circuit 136 may receive a selection of the notification via the user interface 142. Based on the selection, the controller circuit 136 may display the ultrasound image 802.

It should be noted that the various embodiments may be implemented in hardware, software or a combination thereof. The various embodiments and/or components, for example, the modules, or components and controllers therein, also may be implemented as part of one or more computers or processors. The computer or processor may include a computing device, an input device, a display unit and an interface, for example, for accessing the Internet. The computer or processor may include a microprocessor. The microprocessor may be connected to a communication bus. The computer or processor may also include a memory. The memory may include Random Access Memory (RAM) and Read Only Memory (ROM). The computer or processor further may include a storage device, which may be a hard disk drive or a removable storage drive such as a solid-state drive, optical disk drive, and the like. The storage device may also be other similar means for loading computer programs or other instructions into the computer or processor.

As used herein, the term “computer,” “subsystem” or “module” may include any processor-based or microprocessor-based system including systems using microcontrollers, reduced instruction set computers (RISC), ASICs, logic circuits, and any other circuit or processor capable of executing the functions described herein. The above examples are exemplary only, and are thus not intended to limit in any way the definition and/or meaning of the term “computer”.

The computer or processor executes a set of instructions that are stored in one or more storage elements, in order to process input data. The storage elements may also store data or other information as desired or needed. The storage element may be in the form of an information source or a physical memory element within a processing machine.

The set of instructions may include various commands that instruct the computer or processor as a processing machine to perform specific operations such as the methods and processes of the various embodiments. The set of instructions may be in the form of a software program. The software may be in various forms such as system software or application software and which may be embodied as a tangible and non-transitory computer readable medium. Further, the software may be in the form of a collection of separate programs or modules, a program module within a larger program or a portion of a program module. The software also may include modular programming in the form of object-oriented programming. The processing of input data by the processing machine may be in response to operator commands, or in response to results of previous processing, or in response to a request made by another processing machine.

As used herein, a structure, limitation, or element that is “configured to” perform a task or operation is particularly structurally formed, constructed, or adapted in a manner corresponding to the task or operation. For purposes of clarity and the avoidance of doubt, an object that is merely capable of being modified to perform the task or operation is not “configured to” perform the task or operation as used herein. Instead, the use of “configured to” as used herein denotes structural adaptations or characteristics, and denotes structural requirements of any structure, limitation, or element that is described as being “configured to” perform the task or operation. For example, a controller circuit, processor, or computer that is “configured to” perform a task or operation may be understood as being particularly structured to perform the task or operation (e.g., having one or more programs or instructions stored thereon or used in conjunction therewith tailored or intended to perform the task or operation, and/or having an arrangement of processing circuitry tailored or intended to perform the task or operation). For the purposes of clarity and the avoidance of doubt, a general purpose computer (which may become “configured to” perform the task or operation if appropriately programmed) is not “configured to” perform a task or operation unless or until specifically programmed or structurally modified to perform the task or operation.

As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the various embodiments without departing from their scope. While the dimensions and types of materials described herein are intended to define the parameters of the various embodiments, they are by no means limiting and are merely exemplary. Many other embodiments will be apparent to those of skill in the art upon reviewing the above description. The scope of the various embodiments should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. § 112(f) unless and until such claim limitations expressly use the phrase “means for” followed by a statement of function void of further structure.

This written description uses examples to disclose the various embodiments, including the best mode, and also to enable any person skilled in the art to practice the various embodiments, including making and using any devices or systems and performing any incorporated methods. The patentable scope of the various embodiments is defined by the claims, and may include other examples that occur to those skilled in the art. Such other examples are intended to be within the scope of the claims if the examples have structural elements that do not differ from the literal language of the claims, or the examples include equivalent structural elements with insubstantial differences from the literal language of the claims.

What is claimed is:

1. An ultrasound imaging system comprising:
a matrix array probe including a plurality of transducer elements arranged in an array with an elevation direction and an azimuth direction; and
a controller circuit is configured to control the matrix array probe to acquire ultrasound data along first and second two dimensional (2D) planes, the second 2D plane including an anatomical structure, wherein the first 2D plane extends along the azimuth direction and the second 2D plane extends along the elevation direction, the controller circuit being configured to identify when the anatomical structure is symmetric along the second 2D plane with respect to a characteristic of interest and select ultrasound data along the first 2D plane when the anatomical structure is symmetric.

2. The ultrasound imaging system of claim 1, wherein the first 2D plane is automatically selected at a line of symmetrical axis through the second 2D plane.

3. The ultrasound imaging system of claim 1, wherein the controller circuit is configured to notify the user when the anatomical structure is symmetric with the characteristic of interest.

4. The ultrasound imaging system of claim 1, further comprising a display, wherein the controller circuit is configured to generate an ultrasound image based on the select ultrasound data.

5. The ultrasound imaging system of claim 1, wherein the controller circuit is configured to identify when the anatomical structure is symmetric based on a model generated from machine learning algorithms concerning the characteristic of interest.

6. The ultrasound imaging system of claim 1, wherein the controller circuit is configured to adjust the second 2D plane along the azimuth direction until identifying the anatomical structure that is symmetric with respect to the characteristic of interest.

7. The ultrasound imaging system of claim 6, further comprising a user interface, wherein the adjustment of the second 2D plane is based on instructions received from the user interface.

8. The ultrasound imaging system of claim 1, wherein the controller circuit is configured to determine that the anatomic structure is symmetric based on a shape of the anatomic structure.

9. The ultrasound imaging system of claim 1, wherein the controller circuit is configured to determine that the anatomic structure is symmetric based on a position of the anatomic structure relative to a second anatomic structure.

10. The ultrasound imaging system of claim 1, wherein a position of the matrix array probe is adjusted during the acquisition of the ultrasound data.

11. The ultrasound imaging system of claim 1, wherein the first scan plane represents a mid-sagittal plane of a patient.

12. The ultrasound imaging system of claim 1, further comprising a display, wherein the controller circuit is configured to generate a first and second ultrasound image of the ultrasound data of the first and second 2D planes, respectively.

13. A method for selecting a two dimensional (2D) scan plane, the method comprising:
acquiring ultrasound data along first and second 2D planes from a matrix array probe, wherein the second 2D plane includes an anatomical structure, the first 2D plane extending along the azimuth direction and the second 2D plane extending along the elevation direction;
identifying when the anatomical structure is symmetric along the second 2D plane with respect to a characteristic of interest; and
selecting select ultrasound data along the first 2D plane when the anatomical structure is symmetric.

14. The method of claim 13, further comprising notifying the user when the anatomical structure is symmetric.

15. The method of claim 13, further comprising generating an ultrasound image based on the select ultrasound data, and displaying the ultrasound image on the display.

16. The method of claim 13, wherein the identifying operation is based on a model generated from machine learning algorithms.

17. The method of claim 13, further comprising adjusting the second 2D plane along the azimuth direction based on instructions received from a user interface.

18. The method of claim 13, wherein the identifying operation is based on a shape of the anatomic structure.

19. The method of claim 13, wherein the identifying operation is based on a position of the anatomic structure relative to a second anatomic structure.

20. A tangible and non-transitory computer readable medium comprising one or more programmed instructions configured to direct one or more processors to:
acquire ultrasound data along first and second two dimensional (2D) planes from a matrix array probe, wherein the second 2D plane including an anatomical structure, the first 2D plane extending along the azimuth direction and the second 2D plane extending along the elevation direction;
identify when the anatomical structure is symmetric along the second 2D plane with respect to a characteristic of interest; and
select select ultrasound data along the first 2D plane when the anatomical structure is symmetric.
